(19) United States
(12) Patent Application Publication
(10) Pub. No.: US 2026/0306148 A1
(43) Pub. Date: Oct. 8, 2026

(54) Versatile orthopedic implant system with trains and gondolas traveling on a dual traveling track

(71) Applicant: Pampamed Srl, Buenos Aires (AR)

(72) Inventor: Marcelo Hernan Martinez Ferro, Buenos Aires (AR)

(21) Appl. No.: 19/456,173

(22) Filed: Jan. 22, 2026

Related U.S. Application Data
(60) Provisional application No. 63/751,486, filed on Jan. 30, 2025.

(51) Int. Cl. A61B 17/80

(52) U.S. Cl. CPC A61B 17/8023

(57) ABSTRACT

An adaptive versatile orthopedic implant fixation system is provided enabling dynamic conformation to diverse anatomical structures, thereby reducing surgical complexity and improving patient-specific customization. The versatile orthopedic implant system has (a) one or more top travelling devices, referred to as trains, for traveling on a top traveling track, (b) one or more bottom travelling devices, referred to as gondolas, for traveling on a bottom traveling track, and (c) a dual traveling track that comprises of the top traveling track on top of the bottom traveling track.

### CROSS-REFERENCE TO RELATED APPLICATIONS

This application claims priority from U.S. Provisional Patent Application 63/751486 filed Jan. 30, 2025, which is incorporated herein by reference.

### FIELD OF THE INVENTION

This invention relates to orthopedic implant systems

### BACKGROUND OF THE INVENTION

Conventional orthopedic implants present several limitations that can impact their effectiveness in treating fractures, particularly in complex cases. One of the primary drawbacks is their rigid fixation, which may not optimally adapt to all anatomical variations. Additionally, these implants often require intraoperative modifications to fit the specific position and type of fracture, increasing surgical time and complexity. Another significant limitation is their inflexibility in cases of multiple fractures, as they do not always provide the necessary adaptability for managing multiple bone fragments. Furthermore, anatomical incompatibility can arise when implants do not conform precisely to certain bone structures, necessitating additional adjustments or compromising stability.

A critical shortcoming of conventional implants is their single fixation type, which may not be sufficient for complex fractures or patients with unique anatomical or biomechanical challenges. In such cases, new techniques, devices and methods within the same implant may be required to achieve optimal stabilization and promote proper healing. The lack of versatility in existing implant designs highlights the need for new and improved solutions that addresses these challenges effectively.

### SUMMARY OF THE INVENTION

A versatile orthopedic implant system is provided having (a) a top travelling device, referred to as a train, for traveling on a top traveling track, (b) a bottom travelling device, referred to as a gondola, for traveling on a bottom traveling track, and (c) a dual traveling track that comprises of the top traveling track on top of the bottom traveling track. The top traveling path and the bottom traveling path are aligned with each other.

The top traveling track has defined a top traveling path, with a train length defined in line with the top traveling path, and a train width defined orthogonal to the top traveling path. The train has a train hole through the train substantially orthogonal to the top traveling path. Two or more arms expanding laterally and downward from the train body, where downward is defined substantially orthogonal from the top traveling path.

The train has an n-shape body with two train traveling grooves aligned with each other on insides of the n-shape body sized to fit and travel along the top traveling track.

The top traveling track has a top traveling width, where the top traveling track has distributed along the top traveling path sections with widths less than the top traveling width. The train length is designed to bridge each gap created by the less width top traveling path sections so that the two train traveling grooves maintain traction with the top traveling track while traveling.

The bottom traveling track has defined a bottom traveling path, with a gondola length defined in line with the bottom traveling path, and a gondola width defined orthogonal to the bottom traveling path. The gondola has a gondola hole through the gondola substantially orthogonal to the bottom traveling path. Two or more arms expanding laterally and downward from the gondola body, where downward is defined substantially orthogonal from the bottom traveling path.

The gondola has a u-shape body with two gondola traveling grooves aligned with each other on insides of the u-shape body sized to fit and travel along the bottom traveling track.

The bottom traveling track has a bottom traveling width, where the bottom traveling track has distributed along the bottom traveling path sections with widths less than the bottom traveling width. The gondola length is designed to bridge each gap created by the less width bottom traveling path sections so that the two gondola traveling grooves maintain traction with the bottom traveling track while traveling.

In a further embodiment, the top traveling track has top traveling track holes to which the train hole can be aligned and fastened. The bottom traveling track has bottom traveling track holes to which the gondola hole can be aligned and fastened.

The top traveling track and the bottom traveling track can be manufactured as a single dual traveling track.

Embodiments of the invention provide a superior solution to the limitations inherent in conventional orthopedic implants by introducing an adaptable, modular, and multi-fixation design. Existing implants are characterized by rigid fixation mechanisms, requiring intraoperative modifications to accommodate anatomical variations. In contrast, the embodiments described herein incorporate an adaptive fixation mechanism, enabling dynamic conformation to diverse anatomical structures, thereby reducing surgical complexity and improving patient-specific customization.

A distinguishing feature of the embodiments in this invention is the dual traveling track mechanism which allows trains and gondolas to move independently along designated paths. This configuration facilitates precise positioning and fixation at multiple points, ensuring optimal stabilization of complex and multi-fragment fractures. Unlike existing systems, which lack flexibility in addressing multiple bone fragments, the embodiments of the invention enable independent adjustments along the dual track, thereby enhancing mechanical stability and load distribution.

Further, conventional implants suffer from anatomical incompatibility, often necessitating extensive intraoperative modifications to achieve proper fit. The disclosed embodiments eliminate this issue by incorporating a modular and customizable design, allowing for precise adaptation to individual patient anatomy, reducing surgical adjustments, and minimizing implant failure risks.

Additionally, existing orthopedic implants are limited to a single fixation type, restricting their ability to accommodate varying biomechanical requirements. The embodiments of the invention overcome this limitation by supporting multiple fixation methods within a single implant, thereby allowing for a tailored approach to fracture stabilization. This multi-fixation capability ensures enhanced versatility, addressing the diverse needs of orthopedic trauma and reconstructive surgery.

By integrating dynamic adjustability, independent fixation points, and a multi-fixation framework, the embodiments of the invention surpass conventional implants in adaptability, surgical efficiency, and biomechanical performance. The present invention represents a significant advancement in orthopedic implant technology by offering a highly versatile, patient-specific, and structurally optimized solution for fracture management.

### BRIEF DESCRIPTION OF THE DRAWINGS

FIG. 1 shows according to an exemplary embodiment of the invention a versatile and flexible orthopedic implant system.

FIG. 2 shows according to an exemplary embodiment of the invention a train with different view sections.

FIG. 3 shows according to an exemplary embodiment of the invention a gondola with different view sections.

FIG. 4 shows according to an exemplary embodiment of the invention the dual traveling track with different spacer thicknesses.

FIG. 5 shows according to an exemplary embodiment of the invention the dual traveling track with different spacer thicknesses in relation to a traveling train and traveling gondola on their respective tracks.

FIG. 6 shows according to an exemplary embodiment of the invention a top view of a top traveling track.

FIG. 7 shows according to an exemplary embodiment of the invention a top view of a top traveling track with distributed gap sections in relation to train dimensions.

FIG. 8 shows according to an exemplary embodiment of the invention a side view of a top traveling track with distributed gap sections.

FIG. 9 shows according to an exemplary embodiment of the invention a side view of a top traveling track with distributed gap sections in relation to train dimensions.

FIG. 10 shows according to an exemplary embodiment of the invention a top view of a bottom traveling track.

FIG. 11 shows according to an exemplary embodiment of the invention a top view of a bottom traveling track with distributed gap sections in relation to gondola dimensions.

FIG. 12 shows according to an exemplary embodiment of the invention a side view of a bottom traveling track with distributed gap sections.

FIG. 13 shows according to an exemplary embodiment of the invention a side view of a bottom traveling track with distributed gap sections in relation to gondola dimensions.

FIG. 14 shows according to an exemplary embodiment of the invention a side view of a train or gondola with arms on either side of its body to hold an object in between.

### DETAILED DESCRIPTION

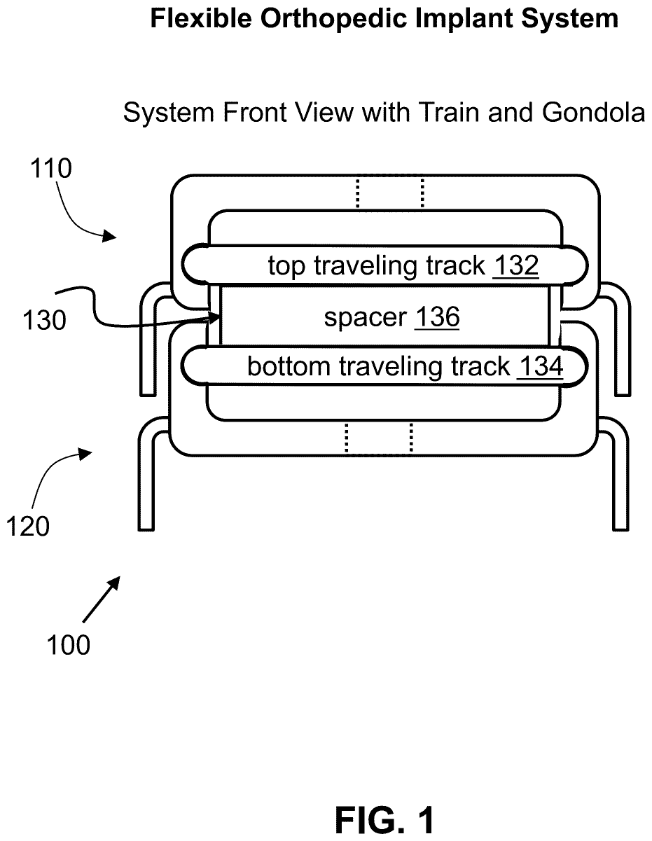

The flexible and versatile orthopedic implant system 100 has three essential components: a top traveling device referred to as a train 110, a bottom traveling device referred to as a gondola 120, and a dual traveling track 130 containing top traveling track 132 and bottom traveling track 134 separated by spacer 136 (FIG. 1).

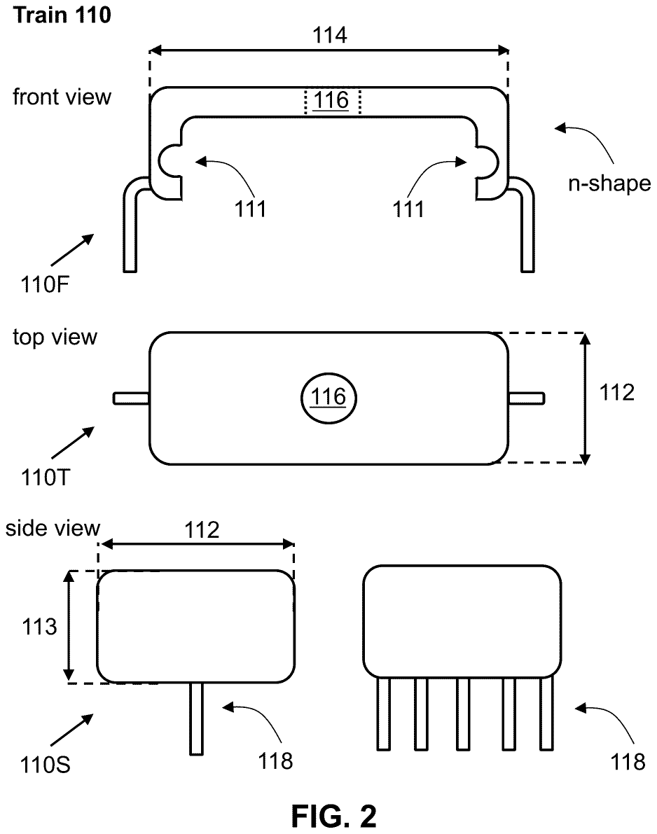

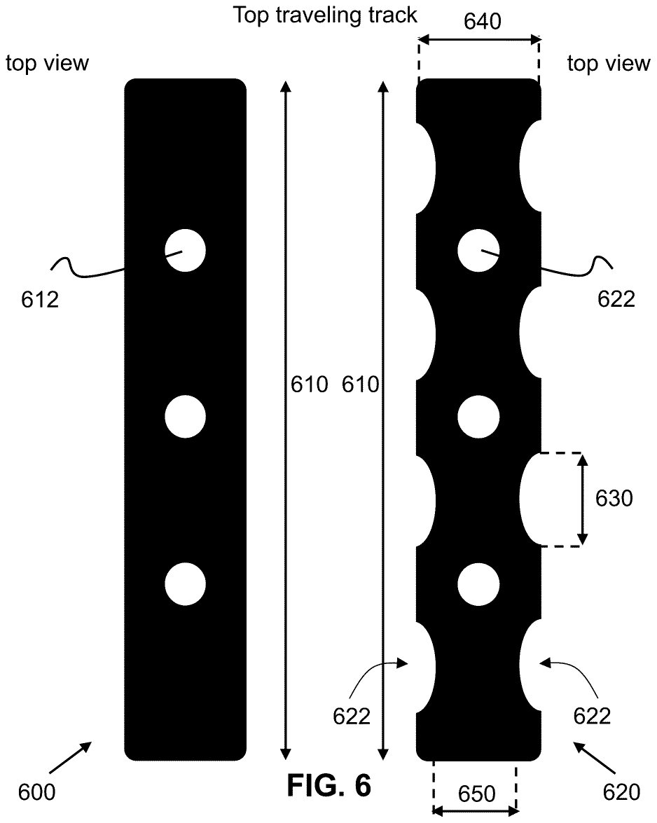

FIG. 2 shows train 110 from three view planes: front view 110F, top view 110T and side view 110S. Train 110 is designed for traveling on top traveling track 132 which has defined a top traveling path 610 (see FIG. 6); the direction train 110 is capable of traveling in either direction of top traveling path 610. Train 110 has a train length 112 defined in line with top traveling path 600, and a train width 114 defined orthogonal to top traveling path 610. A train height 113 is defined for train 110 as can be seen in view 110S.

Train 110 has a train (screw or fastening) hole 116 through train 110 (dashed lines in front view 110F) substantially orthogonal to top traveling path 610. A screw through hole 116 could be used to fasten train 110 to top traveling track 132. This hole can be generalized as a fastening hole where any type of fastener can be used.

Train 110 has two or more arms 118 expanding laterally and downward, where downward is defined substantially orthogonal from top traveling path 610. Noted that in the left diagram of view 110S only one arm of the two arms is shown as the other arm is on the opposite site which is clear in top view 110T. Accordingly, each side of train 110 could have one or more arms as illustrated in side view 110S in FIG. 2. These arms can have a variety of shapes, sizes and materials with the objective to be shaped bend around an object (e.g. a bone, like a rib or sternum) and then to hold that object.

Train 110 has an n-shape body, n-shape, with two train traveling grooves 111 aligned with each other on insides of the n-shape body sized to fit and travel smoothly along top traveling track 310.

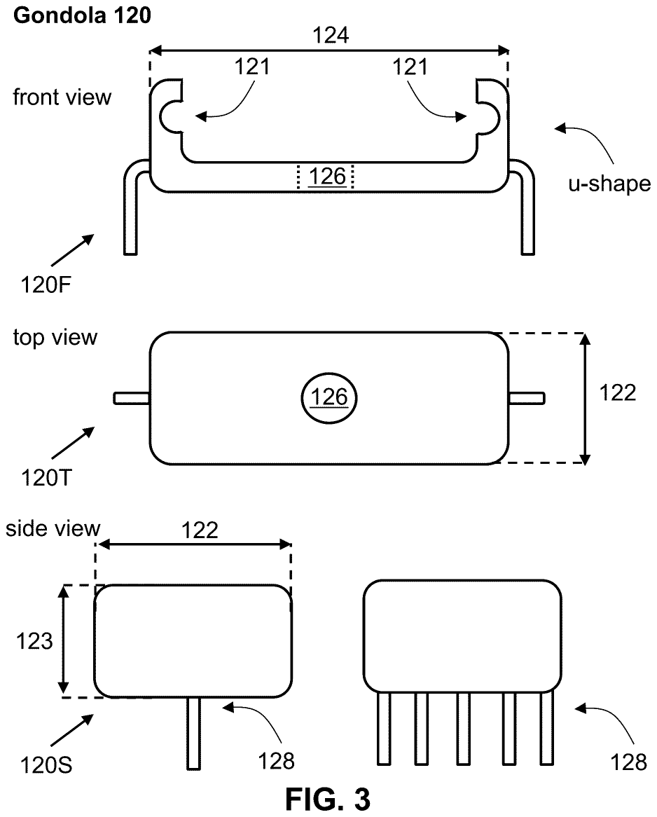

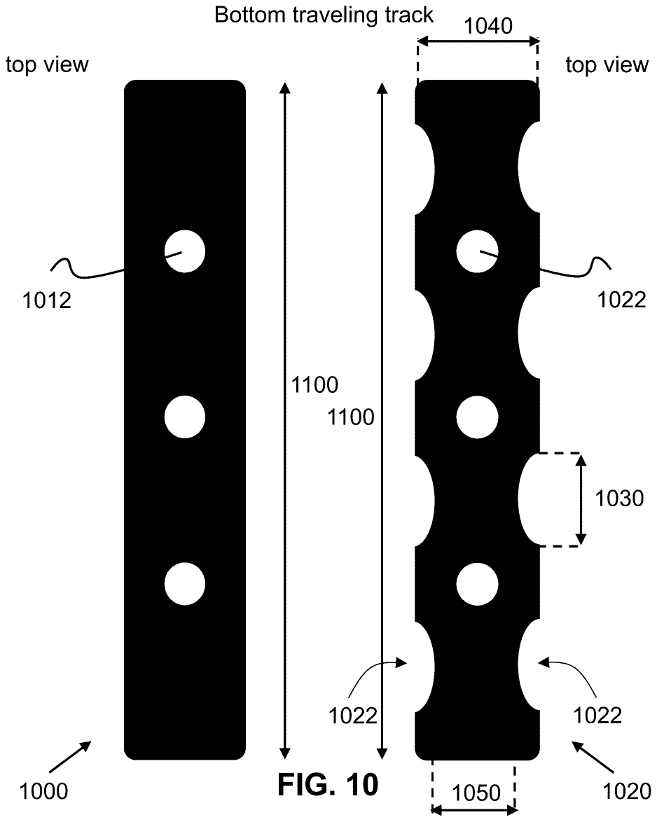

FIG. 3 shows gondola 120 from three view planes: front view 120F, top view 120T and side view 120S. Gondola 120 is designed for traveling on bottom traveling track 134 which has defined a bottom traveling path 1100 (see FIG. 10) the direction gondola 120 is capable of traveling in either direction of bottom traveling path 1100. Gondola 120 has a gondola length 122 defined in line with bottom traveling path 1100, and a gondola width 124 defined orthogonal to bottom traveling path 1100. A gondola height 123 is defined for gondola 120 as can be seen in view 120S.

Gondola 120 has a gondola (screw or fastening) hole 126 through gondola 120 (dashed lines in front view 120F) substantially orthogonal to bottom traveling path 1100. A screw through hole 123 could be used to fasten gondola 120 to bottom traveling track 134. This hole can be generalized as a fastening hole where any type of fastener can be used.

Gondola 120 has two or more arms 128 expanding laterally and downward, where downward is defined substantially orthogonal from bottom traveling path 1100. Noted that in the left diagram of view 120S only one arm of the two arms is shown as the other arm is on the opposite site which is clear in top view 120T. Accordingly, each side of gondola 120 could have one or more arms as illustrated in side view 120S in FIG. 3. These arms can have a variety of shapes, sizes and materials with the objective to be shaped bend around an object (e.g. a bone, like a rib or sternum) and then to hold that object.

Gondola 200 has a u-shape body, u-shape, with two gondola traveling grooves 121 aligned with each other on insides of the u-shape body sized to fit and travel smoothly along bottom traveling track 320.

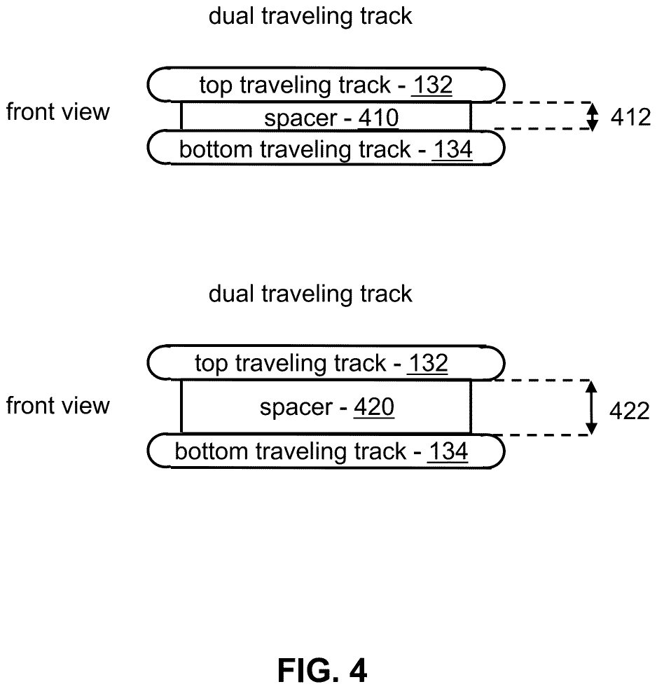

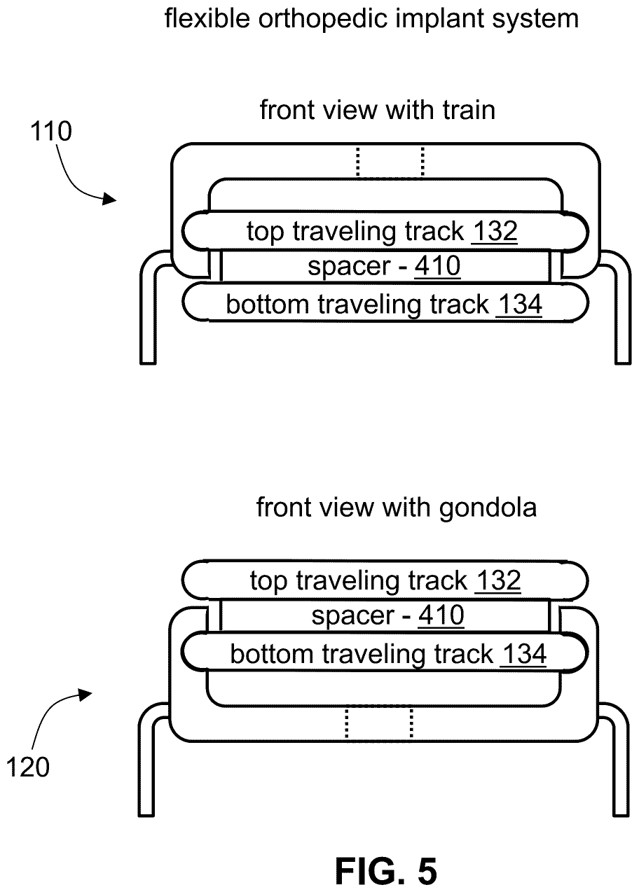

Referring to FIGS. 1 and 4-5 the thickness of spacer can be designed to allow the train and gondola to freely travel along their respective traveling tracks without interfering with each other or running into each other. For example, spacer 420 with a thicker thickness 422 compared to spacer 410 with thickness 412 allows for the train and gondola to pass each other along their respective tracks (see also FIG. 1). FIG. 5 shows a situation in the top part of the figure where train 110 is able to travel on its respective top traveling track 132, but if e.g. train 110 is fastened to its top traveling track 132 then it would limit the range of travel for gondola 120, and vice versa. The reason is that the spacer thickness 412 of spacer 410 (and potentially the body thickness of the train or/and gondola) prevent them from passing each other whole traveling. This is contrast to the embodiment in FIG. 1 and FIG. 4 bottom aspect, where train 110 and gondola 120 are able to freely pass and travel without running into each other mainly controlled by spacer thickness and additionally by body thickness of the train and gondola.

Top traveling track 132 and bottom traveling track 134 are typically aligned with each other. Meaning, top traveling path 610 and bottom traveling path 1100 are aligned with each other as well (see FIGS. 1, 4-5). This allows the train and gondola to travel in similar directions yet on different tracks. In one variation of this embodiment, it would be possible to have both paths not aligned with each other.

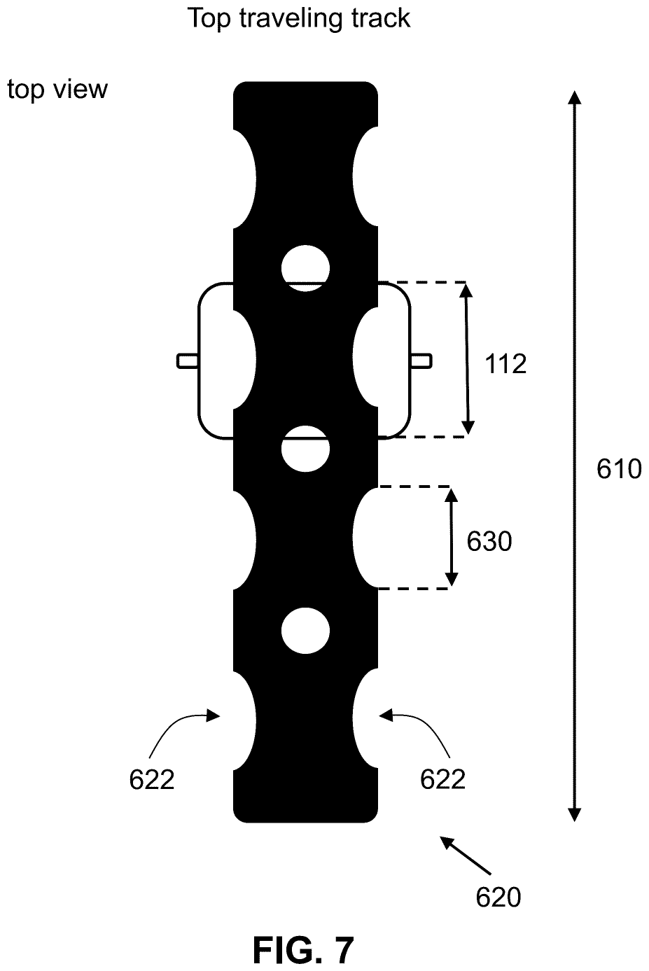

FIG. 6 shows a top view of top traveling track 600 with top traveling path 610 and holes 612 to allow for fastening the trains. Holes 612 can be screw holes or straight holes. FIG. 6 also shows a top view of top traveling track 620 with top traveling path 610 and holes 622 to fasten the trains. Similarly, holes 622 can be screw holes or straight holes. In an alternate embodiment, top traveling track 620 has distributed along its top traveling path 610 sections 622 on either side with widths 650 that are less than top traveling track width 640. Accordingly in an embodiment like that, train length 112 is then designed such that it spans each gap 630 of each of the sections 622 to maintain traction with the top traveling track 620 as shown in FIG. 7.

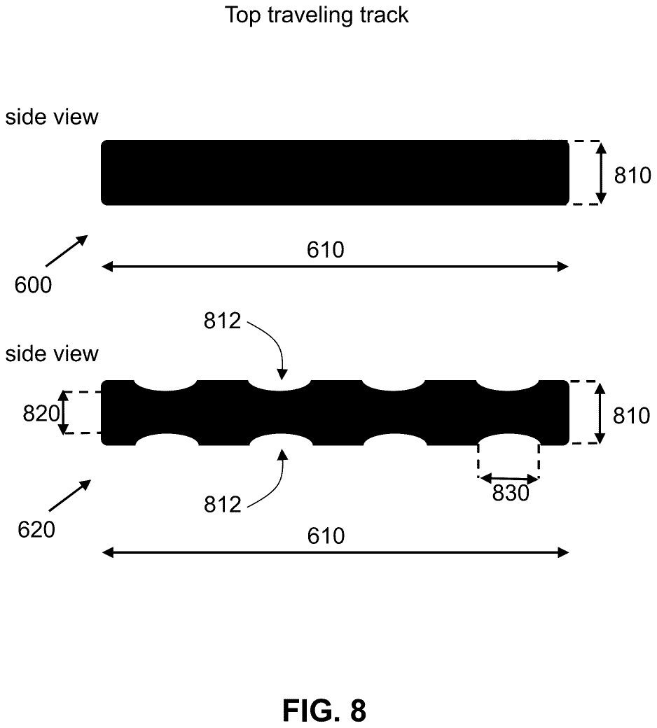

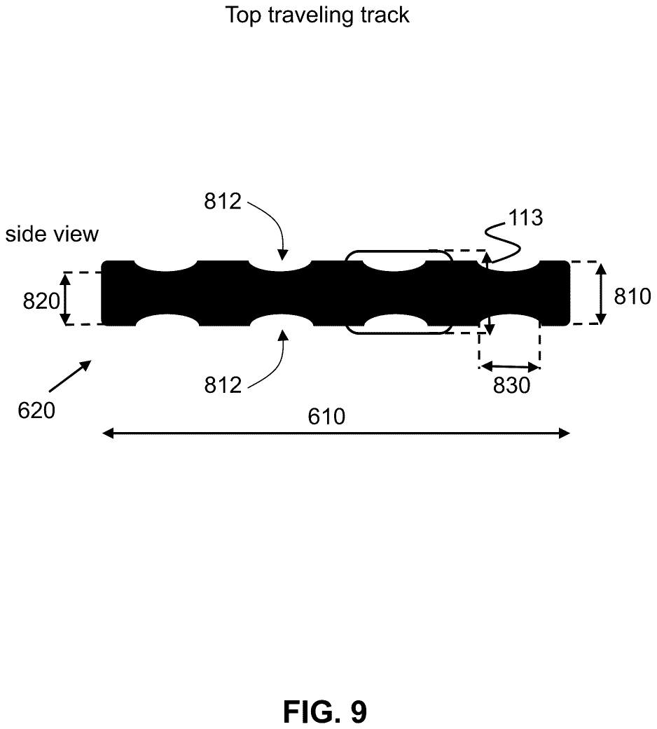

In another alternate embodiment as shown in FIG. 8 side view of top traveling track 620, top traveling track 620 has distributed along top its traveling path 610 sections 812 on either top and/or bottom sides with heights 820 that are less than top traveling height 810. Accordingly in an embodiment like that, train height 113 is then designed such that it spans each gap 830 of each of the sections 812 to maintain traction with the top traveling track 620 as shown in FIG. 9.

The advantage of the thinner sections in height and width is that the overall top traveling track is lighter. In addition, these thinner sections can be designed to allow for quick and easy snapping or cutting to adjust the top traveling track to length.

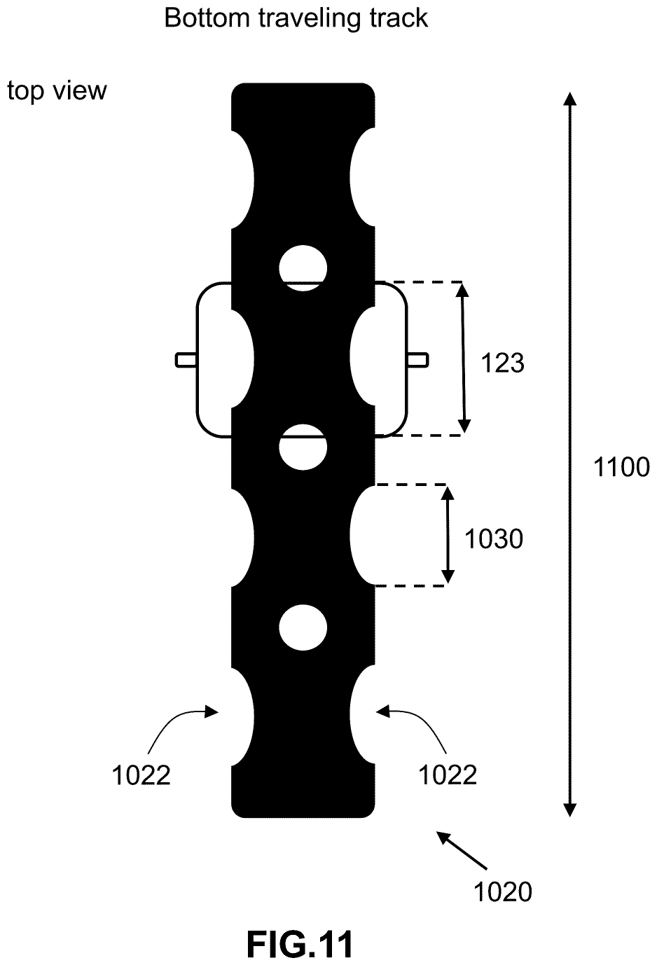

FIG. 10 shows a top view of bottom traveling track 1000 with bottom traveling path 1100 and holes 1012 to allow for fastening the gondola. Holes 1012 can be screw holes or straight holes. FIG. 10 also shows a top view of bottom traveling track 1020 with bottom traveling path 1100 and holes 1022 to fasten the gondola. Similarly, holes 622 can be screw holes or straight holes. In an alternate embodiment, bottom traveling track 1020 has distributed along its bottom traveling path 1100 sections 1022 on either side with widths 1050 that are less than bottom traveling track width 1040. Accordingly in an embodiment like that, gondola length 123 is then designed such that it spans each gap 630 of each of the sections 622 to maintain traction with the top traveling track 620 as shown in FIG. 11.

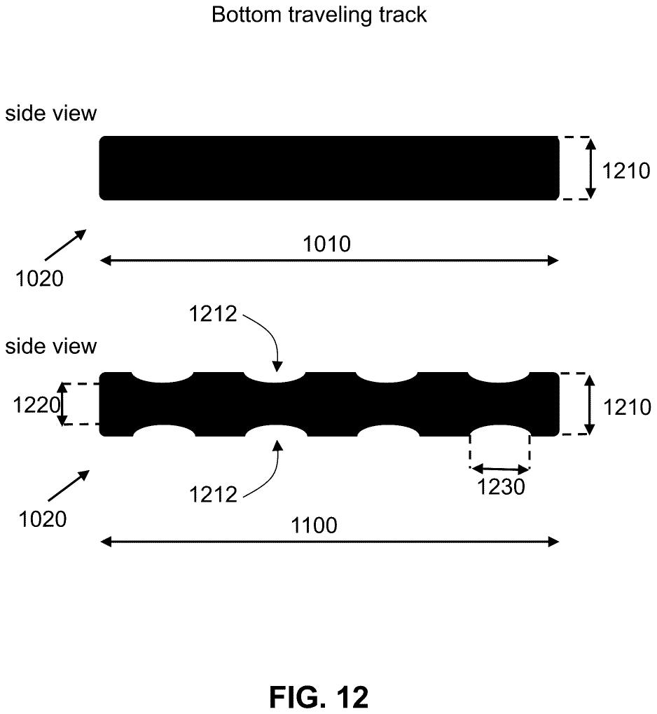

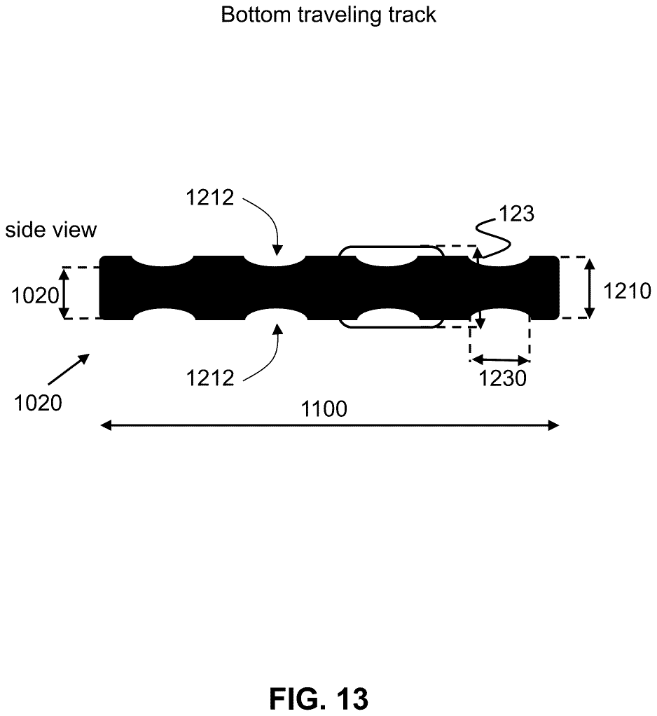

In another alternate embodiment as shown in FIG. 12 side view of bottom traveling track 1020, bottom traveling track 1020 has distributed along its bottom traveling path 1100 sections 1212 on either top and/or bottom sides with heights 1220 that are less than bottom traveling height 1210. Accordingly in an embodiment like that, gondola height 123 is then designed such that it spans each gap 1230 of each of the sections 1212 to maintain traction with the bottom traveling track 1220 as shown in FIG. 13.

The advantage of the thinner sections in height and width is that the overall bottom traveling track is lighter. In addition, these thinner sections can be designed to allow for quick and easy snapping or cutting to adjust the top traveling track to length. In the case that the top and bottom traveling track are aligned with each other, then these sections of less height and/or width can then also be aligned. Fabrication can be done by having separate traveling tracks that are combined in one way or form/shape forming the dual traveling track or they can be manufactured as a single piece forming the dual traveling track. A person skilled in the art would be able to come up with various ways of manufacturing with design objectives like weight, materials, costs or the like.

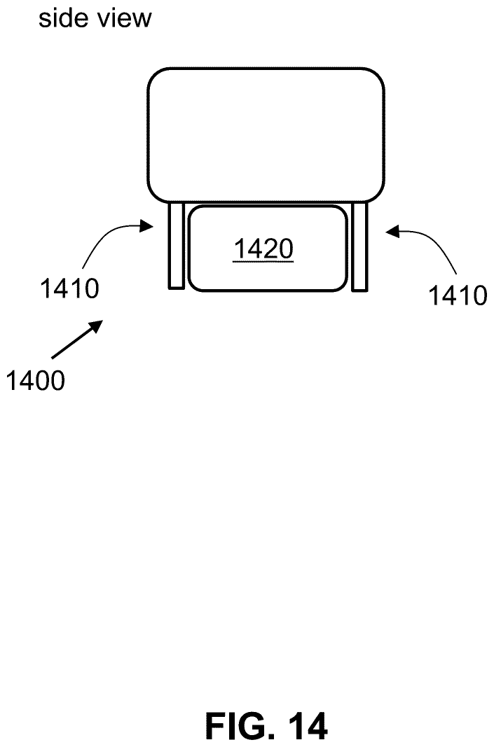

FIG. 14 shows a side view of an embodiment where either 1400 can be a train or gondola with two arms 1410 on either side of its body to hold an object in between (noted that each side has two arms totaling four arms). This object could for example be a bone graft that a surgeon would like to put in place where a gap has occurred along a bone section like e.g. a rib. The train or gondola could then be traveled to its proper location to place the bone graft.

While the train or gondola could travel and be placed along their respective traveling tracks/paths, in an alternate embodiment the train or gondola could also be placed and fastened as the end of their respective paths with a section of their body overhanging their respective traveling track (not shown). This type of train or gondola is then called a caboose.

What is claimed is:

1. A versatile orthopedic implant system, comprising:
(a) a top travelling device, referred to as a train, for traveling on a top traveling track which has defined a top traveling path, wherein the train has a train length defined in line with the top traveling path, and a train width defined orthogonal to the top traveling path, wherein the train has a train hole through the train substantially orthogonal to the top traveling path, and wherein the train has two or more arms expanding laterally and downward, wherein downward is defined substantially orthogonal from the top traveling path;
(b) a bottom travelling device, referred to as a gondola, for traveling on a bottom traveling track which has defined a bottom traveling path, wherein the gondola has a gondola length defined in line with the bottom traveling path, and a gondola width defined orthogonal to the bottom traveling path,
wherein the gondola has a gondola hole through the gondola substantially orthogonal to the bottom traveling path, and
wherein the gondola has two or more arms expanding laterally and downward, wherein downward is defined substantially orthogonal from the bottom traveling path; and
(c) a dual traveling track comprising of the top traveling track on top of the bottom traveling track, wherein the top traveling path and the bottom traveling path are aligned with each other.

2. The versatile orthopedic implant system as set forth in claim 1, wherein the train has an n-shape body with two train traveling grooves aligned with each other on insides of the n-shape body sized to fit and travel along the top traveling track.

3. The versatile orthopedic implant system as set forth in claim 1, wherein the gondola has a u-shape body with two gondola traveling grooves aligned with each other on insides of the u-shape body sized to fit and travel along the bottom traveling track.

4. The versatile orthopedic implant system as set forth in claim 1, wherein the top traveling track has a top traveling width, wherein the top traveling track has distributed along the top traveling path sections with widths less than the top traveling width.

5. The versatile orthopedic implant system as set forth in claim 4, wherein the train length is designed to bridge each gap created by the less width top traveling path sections so that the two train traveling grooves maintain traction with the top traveling track while traveling.

6. The versatile orthopedic implant system as set forth in claim 1, wherein the bottom traveling track has a bottom traveling width, wherein the bottom traveling track has distributed along the bottom traveling path sections with widths less than the bottom traveling width.

7. The versatile orthopedic implant system as set forth in claim 6, wherein the gondola length is designed to bridge each gap created by the less width bottom traveling path sections so that the two gondola traveling grooves maintain traction with the bottom traveling track while traveling.

8. The versatile orthopedic implant system as set forth in claim 1, wherein the top traveling track has top traveling track holes to which the train hole can be aligned and fastened.

9. The versatile orthopedic implant system as set forth in claim 1, wherein the bottom traveling track has bottom traveling track holes to which the gondola hole can be aligned and fastened.

10. The versatile orthopedic implant system as set forth in claim 1, wherein the top traveling track and the bottom traveling track are manufactured as a single dual traveling track.
